(19) United States
(12) Patent Application Publication
(10) Pub. No.: US 2010/0106076 A1
(43) Pub. Date: Apr. 29, 2010

(54) DRUG ADMINISTRATION BASED ON A PATIENT'S ACTIVITY STATUS MEASURED BY ACCELERATION SENSORS

(75) Inventors: Giovanni Nisato, Eindhoven (NL); Roelf Kassies, Eindhoven (NL); Johannes Johanna Van Herk, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/593,123

(22) PCT Filed: Mar. 21, 2008
(86) PCT No.: PCT/IB2008/051082

§ 371 Date: Sep. 25, 2009

(30) Foreign Application Priority Data
Mar. 27, 2007 (EP) 07105001.7

(51) Int. Cl. A61M 31/00; A61N 1/30

(52) U.S. Cl. 604/20; 604/67; 604/66; 604/503

(57) ABSTRACT

It is described a drug delivery system (100) that is adapted to infuse a drug in a profile related to the activity status of a patient (595). The system comprises acceleration sensors (112), dedicated electronics (120) and software to monitor the patient's activity and maintain control over the drug-release profile. The described system (100) enables a personalized treatment of patients (595) by monitoring their individual activity status and coupling this information to a drug delivery following appropriate delivery profiles. The system (100) may be provided with a memory in order to store a time dependence of activity data. This time dependence may be used by physicians to monitor, support and adapt their therapy for the patients. The system (100) maybe realized as a closed-loop system for monitoring and treatment of various diseases such as Parkinson s disease. Accelerations sensors (112) may be used in order to directly monitor a positional state and/or motions of the patient's body. The described system may allow for a more efficient use of the delivered drugs. This will result both in a reduction of the amount of required medication and a reduction of power required by the system (100).

### FIELD OF INVENTION

The present invention relates to the field of delivering drugs to a patient. In particular the present invention relates to a system and to a method for automatically delivering drugs to the body of a patient in a correct dosage by appropriately controlling the operation of a drug delivery device.

### ART BACKGROUND

Traditionally, drug products are given via tablets or oral capsules. These are taken for example once a day in the morning or in the evening. This results in a bolus in the blood drug level after intake and a subsequent exponential decline of the drug in the blood over time. More advanced systems take into account timing of medication to increase drug efficacy by advising the patient to preferably administer the drug at some time of day, for example in the evening after having diner. At some time during the day, prior to the next administration, the blood drug level may be below a lower therapeutic limit and the patient may experience symptoms and/or disease progression.

More and more prophylactic treatment or maintenance therapy is applied in order to combat a disease already at the beginning or before the disease effectively appears. This therapy comprises the administration of lower amounts of drugs at a higher frequency. This results in a smoother, more constant blood drug level being within the therapeutic window.

WO 2006/096654 A2 discloses a microjet fluid delivery system including a reservoir, a delivery actuator and a delivery nozzle having an exit orifice with a diameter between about 1 μm and about 500 μm. The delivery actuator is configured to deliver a quantity of fluid contained in the reservoir into the tissue of a patient. Thereby the fluid has a pre-determined velocity leading to a deposition of the fluid in a desired depth in the tissue. The quantity of fluid may contain one or more therapeutic agents such as medications, drugs, bio-reactive agents, etc. The delivery actuator may also be configured to repeatedly deliver a quantity of the fluid contained in the reservoir through the nozzle at pre-determined intervals. The delivery actuator may be coupled to a sensor such that the quantity of delivered fluid can depend on a signal provided by the sensor. The sensor may be a biosensor selected from one or more of a motion sensor, a pressure sensor, a density sensor, a chemical sensor and/or an electrical sensor. The biosensor may be located internally or externally of a patient's body.

WO 2004/012796 A1 discloses a delivery device suitable for treatment of diseases or conditions in which a drug has to be applied through the skin of a patient. The delivery device is provided for delivering a liquid drug into the body of a patient. The delivery device comprises a reservoir having, in a situation of use, an outlet, an amount of a liquid drug contained in the reservoir, expelling means for expelling the drug out of the reservoir through the outlet, and actuating means for actuating the expelling means. Upon actuation the expelling means is adapted for expelling the drug contained in the reservoir during a period of approximately 7-9 hours. The disclosed drug delivery device has the disadvantage that, once activated, there is no further control of the amount of drug, which is going to be delivered to the patient.

US 2003/104982 A1 discloses a system and a method for dosing a hormone regulating the blood glucose, especially insulin for a diabetic patient. In order to improve the administration of the hormone, the invention provides the following characteristic combination: (a) a measuring device to detect measured values correlatable with blood sugar, (b) a controlling means comprising a controller and a hormone dosing device for supplying a hormone dosage, (c) a pilot control device acting on the hormone fine dosage controlling means for performing a coarse pre-control in accordance with at least one influence variable that influences blood glucose.

There may be a need for providing a system for delivering drugs into the body of a patient, which system allows for an appropriate control of the amount of drug being delivered to a patient.

### SUMMARY OF THE INVENTION

This need may be met by the subject matter according to the independent claims. Advantageous embodiments of the present invention are described by the dependent claims.

According to a first aspect of the invention there is provided a system for delivering at least one drug to the body of a patient. The provided system comprises (a) a measurement device for measuring at least one value of a parameter, which is indicative for the activity status of the patient, (b) a control unit, which is coupled to the measurement device and which is adapted to determine a control signal based on the value of the parameter, and (c) a drug delivery device, which is coupled to the control unit and which is adapted to deliver drugs to the patient's body based on the control signal. Thereby, the measurement device comprises a plurality of acceleration sensors.

This aspect of the invention is based on the idea that in many cases the required amount of drug administration to a patient's body strongly depends on the activity status of the patient, wherein the activity status is determined by a motion pattern respectively by the movement status of the patient's body. By measuring this motion pattern by means of at least two acceleration sensors, the current motion of different parts of the patient's body can be measured.

The described drug dosage dependency on the activity status can be used in order to optimally adjust the drug dose. This may provide the advantage that a disease the patient is suffering from can be treated in an optimal way. Therefore, depending on the particular disease the comfort of the patient and/or the expected lifetime of the patient may be increased. The administration of the optimal drug dosage further provides the advantage that the amount of drug being necessary for treating a disease can be reduced such that the effective medical costs for the treatment can also be reduced.

The described system may be used for instance for measuring tremor motions of a patient suffering from Parkinson's disease. By controlling the drug delivery device based on the strength and/or the frequency of the tremor motions a closed-loop system for treating the Parkinson's disease can be established. Therefore, the described system enables a personalized treatment of patients by directly coupling tremor sensing information to drug delivery following on-demand and/or programmed delivery profiles.

The expression “activity status” has to be understood in a very general way. Thereby, the activity may be caused by an active control of the patient. However, activity may also be caused by symptoms a patient is suffering from. In particular, in the case of Parkinson's disease tremor motions representing a disease symptom are also considered to contribute to the activity status.

The at least two acceleration sensors may be coupled with each other such that a sensor array is formed, which is capable of effectively monitoring also complicated motions of the patient's body within three spatial dimensions. Therefore, the acceleration sensors may represent a tremor detection system, which can provide detailed information about the current status of for instance a Parkinson's disease patient.

In particular when tremor motions are supposed to be sensed the acceleration sensors should be placed at appropriate positions. Therefore, it can be advantageous that (a) the measurement device respectively the acceleration sensors and (b) the control unit respectively the drug delivery device are separate preferably modular devices.

The system may be realized by means of an integral device, which is adapted to accomplish the function of at least two of the above-mentioned devices, namely the measurement device, the control unit and/or the drug delivery device. By contrast thereto, the system may also be realized by separate devices, which are coupled with each other in an appropriate way. In particular separate acceleration sensors may be used for sensing tremor motions individually at different positions of the patients body. The coupling between the different components of the described system can be realized by means of a wired or a wireless connection.

The drug delivery device may be used to deliver one type of drugs. However, the drug delivery device may also be adapted to deliver two or more different types of drugs in a controlled manner. Thereby, the administration of different types of drugs may be carried out sequentially or in combination, wherein the ratio of ingredients can be adjusted.

It has to be mentioned that the described system for delivering drugs is applicable not only for human patients but also for animals. In particular in affluent societies there is an increasing demand for a treatment of animals respectively domestic animals, which suffer from similar diseases as human beings.

According to a further embodiment of the invention the control signal is a function of at least one measurement value. Thereby, the measurement value may be evaluated based on the sensor signal being provided by the at least two acceleration sensors.

According to a further embodiment of the invention the control unit is adapted to determine the control signal in real time. This may provide the advantage that the whole drug delivery process can be carried out in real time with respect to the current activity status respectively the current motion status sensed by the at least two acceleration sensors. Thereby, the drug delivery can be accomplished in a continuous or at least in a quasi-continuous manner such that by contrast to a discrete drug administration it is much easier to keep the drug dosage within or at least close to the optimal therapeutic range.

According to a further embodiment of the invention the control unit is adapted to determine the control signal based on the current time of day. This may allow for an optimal drug dosage in particular if, for instance because of medical reasons, the appropriate dosage varies during the course of the day.

According to a further embodiment of the invention the system further comprises a memory for storing a plurality of values of the parameter, which values are indicative for the activity status of the patient. Thereby, the values have been acquired at different times. In particular in case of Parkinson's disease the activity status may be represented by the degree of motion symptoms.

This may provide the advantage that a typical daily rhythm of the patient's activity in particular with respect to a diurnal or a nocturnal profile can be monitored. Thereby, time averaged activity data can be used as an objective measurement to get knowledge when the patient is asleep or at least at rest for some time.

Based on the daily rhythm, which can be measured for instance by measuring the core body temperature (alone or in combination with another measurement), the drug dosage may be adjusted. A low body core temperature may be used as an indicator that the patient is asleep and the drug delivery may be temporarily stopped. The drug delivery may be continued when the patient awakes or just prior to the awakening of the patient. In this respect the drug delivery device may communicate with an alarm clock used by the patient to wake up in the morning. The delivery can start a certain time prior to the alarm time set by the patient. This means that the wake-up time the patient programs in the alarm clock will be used to determine the start of medication delivery prior to wake-up.

It has to be mentioned that such a deactivating of the drug delivery device may also be prevented if the patient can turn on a so-called “sleep mode” on the drug delivery device. This can ensure that the drug delivery device will not be switched off and drug delivery is guaranteed.

In case of a patient suffering from Parkinson's disease the memory may be used for storing data representing the intensity of tremor motions as a function of time. This may provide the advantage that in connection with different drug delivery states the patient's response to the drug administration can be studied when the patient is situated in a familiar environment such as the home of the patient. This may reduce an extra stress, which could influence the outcome of such types of tests.

Further, in case of a patient suffering from Parkinson's disease the memory may store data, which represent the daily rhythm of the patient's activity. Therefore, it is possible to initiate an enhanced drug delivery for instance already two hours before the patient wakes up. This may further improve the treatment of the patient's Parkinson disease.

The amount of drug administration can be controlled by using stored measurement data representing the activity status respectively the motion status of the patient's body, which status has been monitored within a predetermined period of time prior to the current time of day. This means that the administered drug dosage does not only depend on the current activity status, the administered drug dosage may rather depend on the average activity status within a certain span of time preceding the current point in time.

According to a further embodiment of the invention the system is adapted to store drug delivery data in the memory. This may provide the advantage that a physician will be able to exactly recall the drug administration dose as a function of time. By comparing these drug dosage profiles with measured data representing the intensity of tremor motions a physician will be able to adapt the disease treatment individually for each patient.

However, the memory may also be used in order to store pre-programmed drug delivery profiles. Such pre-programmed drug delivery profiles may comprise parameters, which influence the drug dosage, which is supposed to be administered in the future.

According to a further embodiment of the invention the system further comprises a unit for evaluating the frequency distribution of a signal provided by the acceleration sensor.

Since the frequency distribution of tremor motions of patients suffering from Parkinson's disease may contain valuable information about the actual state of the disease, the tremor motion monitored in the frequency domain may provide further information to a physician in order to select appropriate measures for the treatment of Parkinson's disease. In order to convert the time domain signals provided by the acceleration sensors into a frequency domain, known techniques such as for instance a Fast Fourier Transformation may be employed. Alternatively, direct real-time autocorrelation techniques can be used to detect tremor motions.

According to a further embodiment of the invention the measurement device, the control unit and/or the drug delivery device are designed to be externally and mechanically coupled to a patient's body. This may provide the advantage that the drug delivery system can be easily used without having the need of performing at least a minimal invasive operation of the patient, wherein the system or parts of the system are implanted. Of course, it will be necessary to connect at least a portion of the drug delivery device with the patient's body. However, such a connection can already be realized by means of a small injection needle, which can easily penetrate the skin of the patient's body. Of course, also needle less injection techniques can be used for transferring a drug into the patient's body.

The described drug delivery system can be realized as a modular system comprising devices, which can be easily replaced by corresponding devices having the same or a similar function. These functions are diagnosis, monitoring and treatment of a particular disease. In particular different types of sensors can be used as a measurement device such that different physical and/or chemical indicators can be used for controlling the drug release performed by the drug delivery device.

The described modular drug delivery system can be used in particular for the monitoring and the treatment of Parkinson's disease. Thereby, different types of acceleration sensors may be used. In particular three-dimensional acceleration sensors can be used for directly detecting a tremor motion. A network, preferably wireless, of such sensors can be implemented to detect dyskenisia or fall of the patient.

However, in addition to the acceleration sensors also other types of sensors can be employed such as a heart monitoring sensor and or a skin impedance sensor. By simultaneously using different types of sensors complementary information regarding the current symptoms of a particular disease can be obtained, which complementary information may significantly improve the data base of the described drug delivery system. Therefore, both the diagnosis and the treatment of the patient's disease might become more effective.

The described system provides a platform for a personalized treatment of patients suffering from Parkinson's disease. In particular when at least one acceleration sensor is used the described system may combine quantitative tremor motion sensing and a user input to optimize the flux of drug being delivered. Therefore, the system has the potential to optimize Parkinson's management with all kind of drugs, which might be applicable for a treatment. This may delay the need for invasive procedures.

In this respect it has to be mentioned that new methods that increase the cost effectiveness and quality of life have a higher chance of being accepted by patients, by practitioners and also by insurance companies. Since a more continuous dosing of drug is expected to significantly reduce side effects, the described drug delivery system may make some drugs unnecessary, which are currently used to suppress side effects. Therefore a more effective treatment method taking benefit from the described system may lead to a cost saving for treating Parkinson's disease.

According to a further embodiment of the invention the measurement device, the control unit and/or the drug delivery device are wearable devices. Preferably, the wearable devices can be attached to articles of clothing. The attachment can be realized by using various fastening means. For instance hook-and-loop-fasteners (Velcro fasteners) and/or button pockets can be used for detachably attach a wearable device. Further, the attachment can also be realized for instance by means of hypoallergenic skin adhesives such as polyurethane based materials.

Esthetic aspects such as a miniaturization of the devices, personalized design, different color plastic caps in order to ensure a fit with different types of clothing can be taken into account. This may have the advantage that compliance of patients for using the described drug delivery system will be increased.

According to a further embodiment of the invention the coupling between the measurement device, the control unit and the drug delivery device is at least partially realized by means of a wireless connection. This may provide the advantage that the various components of the described system can be placed at the patient's body without having the hassle of taking care about wires which connect the various system components.

According to a further embodiment of the invention the drug delivery device is realized by a module, which can be spatially separated from the measurement device and/or from the control unit. This may provide a high flexibility because the drug delivery device can be attached to an optimal position at the patient's body.

According to a further embodiment of the invention the drug delivery device is a micro jet based device. This means that a high-pressure micro-jet of liquid drug can be generated, which is capable of penetrating the patient's skin. This may provide an in particular elegant way to transfer a liquid drug through the patient's skin into the patient's body. Since no needles have to be used the risk for causing infections can be significantly reduced. It has to be mentioned that apart from micro jet based devices also other drug delivery devices such as infusion pumps may be used.

According to a further embodiment of the invention the drug delivery device is an iontophoretic device.

Iontophoretic devices, which exploit the well-know effect of iontophoresis, allow for non-invasively propelling a charged drug transdermally by repulsive electromotive force using a small electrical charge applied to an iontophoretic chamber containing a similarly charged active agent and its vehicle. Thereby, one or two chambers are filled with a solution containing an active ingredient and its solvent, termed the vehicle. The positively charged chamber, termed the anode will repel a positively charged chemical, whilst the negatively charged chamber, termed the cathode, will repel a negatively charged chemical into the skin.

According to a further embodiment of the invention the measurement device comprises a magnetic field sensor.

By evaluating the time dependency of the strength and/or of the orientation of a magnetic field it is possible to obtain a further measure for the activity status of a patient. Thereby, more detailed information about the activity status may be obtained from evaluating the static posture of the patient and/or from evaluating the dynamic of changes of the strength and/or the orientation of the magnetic field such that information about the dynamic movement of the patient can be obtained. The magnetic field, which is used for determining the activity status, may be represented by all kinds of magnetic fields which are existent in and around the patient. Since the sensitivity of magnetic sensors is quite high also the earth magnetic field may be sensed in order to obtain information about the static posture and/or the dynamic motion of the patient.

According to a further embodiment of the invention the measurement device comprises a temperature sensor. This means that also the body temperature might be used as a further measure for the activity status of the patient's body. Thereby, the temperature can be measured in the core and/or at the surface of the patient's body. Also the temperature of the patient's skin can provide valuable information regarding the activity status.

According to a further embodiment of the invention the measurement device comprises a skin impedance sensor. This means that the electric resistivity may also be used as a further measure for the activity status of the patient's body. Thereby, the resistance respectively the impedance can be measured under direct current and/or under alternating current conditions. The resistance may be affected in particular by the humidity and/or the salt concentration at the skin surface. Therefore, also the impedance of the patient's skin can provide valuable information regarding the patient's activity status.

According to a further embodiment of the invention the measurement device comprises a heart rate sensor and/or a sweat sensor. This may provide the advantage that these types of sensors can give alone or in combination a comparatively precise impression of the stress level the patient is exposed.

According to a further embodiment of the invention the system further comprises a communication unit, which is coupled to the control unit. The provision of an external communication unit may allow for a communication between the described drug delivery system and an external system providing control information such as for instance an external control signal. Further, measurement data may be received by the external system. The measurement data may be evaluated in particular by a physician in order to obtain more detailed information about the status of the patient's disease.

The communication between the external system and the drug delivery system may be carried out by an appropriate cable connection or in a wireless manner. In the latter case the communication unit may have to be coupled with an antenna system.

The provision of an external communication unit may provide the possibility that the drug delivery system can communicate with a medical alert system. This has the advantage that in case of a serious detected event such as a fall down, the alert system can initiate appropriate measures in order to help the patient.

The external system and/or a memory being coupled with the control unit may store tremor data and/or delivery profile data. These data may be of great value for practitioners to monitor, support and adapt their therapy for the patient's suffering for instance from Parkinson's disease. The external system may be for instance a so-called remote patient monitoring system.

According to a further aspect of the invention there is provided a method for delivering drugs to the body of a patient. The method comprises (a) measuring at least one value of a parameter by means of a plurality of acceleration sensors (812), which parameter is indicative for the activity status of the patient (895), (b) determining a control signal based on the value of the parameter, and (c) delivering drugs to the patient's body based on the control signal.

This aspect of the invention is based on the idea that the required amount of a drug administration to a patient's body may strongly depend on the activity status of the patient, wherein the activity status is characterized by a motion pattern respectively a movement status of the patient's body or at least of parts of the patient's body. By measuring the activity status of the patient this dependency can be used in order to optimally adjust the drug dose, which has to be delivered. This may provide the advantage that a disease the patient is suffering from can be treated in an optimal way. The optimal drug dosage may provide the advantage that the amount of drug being necessary for treating a disease can be reduced such that the effective medical costs for the treatment can also be reduced.

In particular in case of Parkinson's disease the activity status may be represented by the degree of motion symptoms.

It has to be noted that embodiments of the invention have been described with reference to different subject matters. In particular, some embodiments have been described with reference to apparatus type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

### BRIEF DESCRIPTION OF THE DRAWINGS

FIG. 1 shows a simplified block diagram of a drug delivery system comprising a motion sensing unit and both an input device and an output device.

FIG. 2 shows perspective views of wearable miniaturized modules each comprising a sensing unit, a power supply, a control unit and a transmission unit, which modules are designed in a button like shape.

FIG. 3a shows a diagram depicting time dependency of tremor caused acceleration, which has been measured by employing a miniaturized module shown in FIG. 5.

FIG. 3b shows a diagram depicting the Fourier Transform of the amplitude of the tremor acceleration shown in FIG. 6a.

FIG. 4 shows a wearable micro-jet drug delivery device comprising straps for conveniently attaching the micro jet drug delivery device to a patient's body.

FIG. 5 shows a drug delivery device and various motion sensing devices being attached at various locations of the body of a patient and exhibiting a sensor network for sensing tremor and posture of the patient.

### DETAILED DESCRIPTION

The illustration in the drawing is schematic. It is noted that in different figures, similar or identical elements are provided with the same reference signs or with reference signs, which are different from the corresponding reference signs only within the first digit.

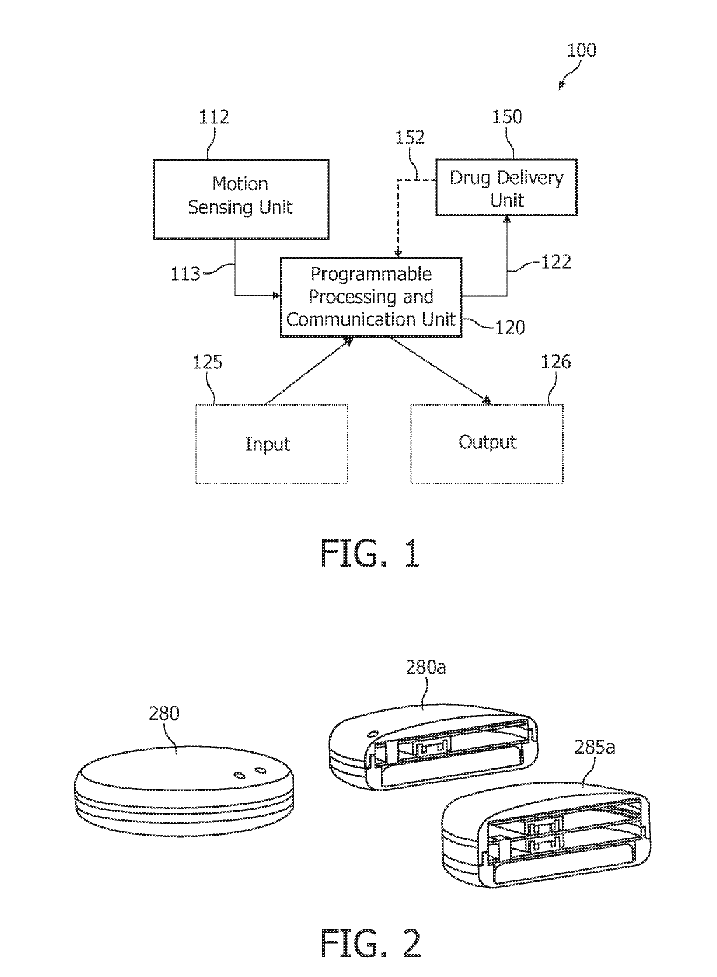

FIG. 1 shows a simplified block diagram 100 of a drug delivery system according to another preferred embodiment of the invention. The drug delivery system 100 is in particular suitable for monitoring and treatment of a patient suffering from Parkinson's disease.

The drug delivery system 100 comprises a motion sensing unit 112, which is coupled to a control unit 120 by means of a control line 122. The control line 122 may be either a wired connection or a wireless connection. According to the embodiment described here, the motion sensing unit comprises a 3D acceleration sensor 112, which is capable of sensing tremor motions of the patient. In order to be able to reliably detect different tremor motions, the motion sensing unit 112 is designed such that it can be detachably affixed to different parts of the patient's body.

The control unit is realized by means of a programmable processing and communication unit 120, which comprises an input device 125 and an output device 126. The input device 125 may be used for receiving external control signals provided for instance by the patient, by a medical person such as a physician. The output device 126 may be used for providing data such as measurement or control data of the drug delivery system (a) to the patient, (b) to a medical person such as a physician and/or (c) to an external system such as for instance a so-called remote patient monitoring system. This makes it easier for a physician to obtain detailed information about the status of the patient's disease.

The drug delivery system 100 further comprises a drug delivery device 150, which is coupled to the control unit 120 by means of a control line 122. Also the control line 122 can be realized by means of a wired or a wireless connection. A feedback line 152, which extends between the drug delivery device 150 and the control unit 120, guarantees a precise operation of the drug delivery device 150.

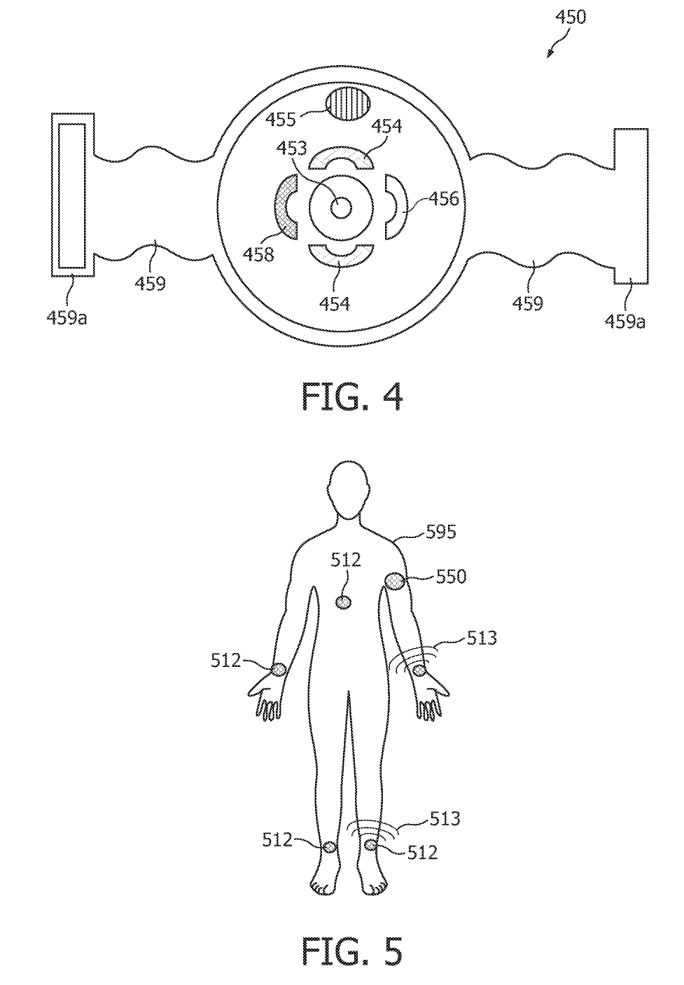

All components of the drug delivery system may be realized as wearable devices, which can be conveniently attached to the patient's body. This also holds for the drug delivery device 150, which may be a so-called micro-jet device for transferring liquid drug through the skin of the patient. An appropriate micro jet device will be described below with reference to FIG. 4.

According to a preferred embodiment of the invention the processing and external communication unit 120 is capable of performing a real-time signal analysis of at least one motion sensing device 112. The motion sensing device 112 comprises a plurality of individual acceleration sensors, which in combination represent an acceleration sensor array. In particular if the acceleration sensors of such an array are attached to different parts of the patient's body, whereby such an array may allow for a reliable, precise and/or quantitative detection of Parkinson's related body signs. These body signs include tremor, rigidity, loss of balance etc.

The control unit 120 is capable of performing a real-time and/or frequency domain analysis of tremor motions. Thereby, the presence of the tremor and its intensity can be detected in a quantitative way.

The control unit 120 may be adapted to store patient drug delivery profiles, which can be used to give feedback on compliance and usage patterns. Further, the efficiency of the treatment of a disease can be monitored in an efficient manner. Thereby, the evolution of the disease can be monitored in a quantitative manner. The drug delivery system 100 may further allow for a closed loop monitoring and treatment, which can be carried out either by the patient, by the caregiver and/or in an automated way.

For example, in the case of a loss of balance and/or the detection of an increased tremor activity, a signal to the used may be provided, which comprises the advice to increase the drug dose. In case of a detection of a stress state a signal can be provided to the user, which signal comprises the advice to reduce or to increase the drug dose. If the system detects that the patient is asleep a reduction of the drug dose may be indicated. Thereby, for detecting whether the patient is asleep, the time of the day may also be taken into account. Of course, the drug dose may be increased prior to a wakeup of the patient.

In case of serious detected events such as a fall, the drug delivery system 100 may be interfaced with an alert system (e.g. MedLine) in order to allow for a rescue of the patient.

The wearable drug delivery device 150 may be based on a tuneable, controllable drug delivery system with start/stop capabilities. The drug delivery device 150 may be realized by an infusion pump, iontophoretic devices or micro jet based devices. Preferably, the drug delivery device 150 is capable of readily (<minutes) adjusting or stopping the delivery rate of medication to readily respond to the input of the user or the automated processing system. Medications, which can be delivered with the described drug delivery system 100 are for example dopamine receptor agonist compounds such as apomorphine.

FIG. 2 shows perspective views of wearable miniaturized modules 280, 280a and 285a. Each of these modules comprises a sensing unit, a power supply, a control unit and a transmission unit. In order to increase the compliance of patients the modules 280, 280a and 285 are designed in a button like shape. This ensures that the module can be easily inserted into pockets of a patient's clothing. In FIG. 2, reference numeral 280 denominates a perspective view and reference numeral 280a denominates a perspective cross sectional view of a module, which comprises the necessary electronic circuitry on a single printed circuit board. Reference numeral 285a denominates a perspective cross sectional view of a module, which comprises two printed circuit boards carrying the necessary electronic circuitry of the module.

In order to increase patient compliance, the modules are easy to use and unobtrusive. The modules can be worn with Velcro-fastened bracelets. Ideally they could be integrated in clothing for instance with Velcro fasteners or buttons and/or they could be inserted into pockets.

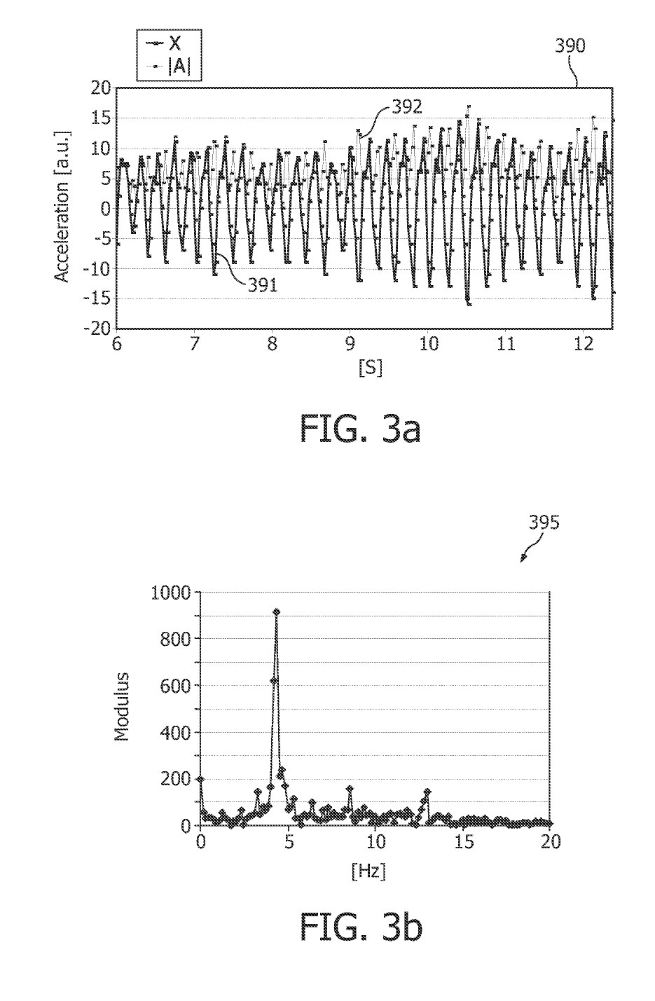

FIG. 3a shows a diagram 390 depicting time dependency of tremor caused acceleration, which has been measured by employing the miniaturized module 280 shown in FIG. 2. Thereby the curve 391 represents the measured acceleration amplitude in an x-direction. The curve 392 represents the absolute value of the acceleration amplitude. FIG. 3b shows a diagram 395 depicting the Fourier Transform of the amplitude 391 of the tremor acceleration shown in FIG. 3a. An evaluation of the frequency domain curve 395 may help a physician to conveniently analyze the tremor motion.

FIG. 4 shows a wearable micro-jet drug delivery device 450. The micro jet drug delivery device 450 is realized in a miniaturized form such that it can be easily attached to the patient's body without reducing the comfort of the patient. Therefore, the micro-jet drug delivery device 450 is equipped with straps 459, which can be attached to each other or to a piece of clothing of the patient by means of fixations means 459a. According to the embodiment described here, the fixation means are Velcro fasteners 459a.

The described micro jet drug delivery device 450 comprises a micro jet injector 453, which is capable of generating a high speed micro beam of drug which can penetrate the patient's skin and enter the patient's body without using an injection needle. In order to monitor the correct position of the drug delivery device 450 onto the patient's skin, skin contact sensors 454 are provided. Further, there is provided a temperature sensor 456 and an irritation sensor 458 for providing further complementary information for the drug delivery system. This complementary information may also be used for controlling the operation of the micro jet injector 453. An USB port is provided for allowing communication with a not depicted external device.

FIG. 5 shows a drug delivery device 550 and various motion sensing devices 512 being attached at various locations of the body of a patient 595. The motion sensing devices, which according to the embodiment described here are 3D acceleration sensors respectively 3D posture sensors 512, represent a sensor network for sensing tremor and posture of the patient 595. The motion sensing devices 512 can be attached at appropriate locations at the patient's body respectively the patients clothing.

The described drug delivery system can be used in particular for a comprehensive Parkinson's disease management, which includes the diagnostic and the treatment of Parkinson's disease. The compliance and the evolution of Parkinson's disease can be monitored. Further, the described drug delivery system can be used as a feedback loop system management of Parkinson's disease either involving human intervention or an automated closed loop.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

In order to recapitulate the above described embodiments of the present invention one can state: It is described a drug delivery system 100 that is adapted to infuse a drug in a profile related to the activity status of a patient 595. The system comprises acceleration sensors 112, dedicated electronics 120 and software to monitor the patient's activity and maintain control over the drug-release profile. The described system 100 enables a personalized treatment of patients 595 by monitoring their individual activity status and coupling this information to a drug delivery following appropriate delivery profiles. The system 100 may be provided with a memory in order to store a time dependence of activity data. This time dependence may be used by physicians to monitor, support and adapt their therapy for the patients. The system 100 may be realized as a closed-loop system for monitoring and treatment of various diseases such as Parkinson's disease. Accelerations sensors 112 may be used in order to directly monitor a positional state and/or motions of the patient's body. The described system may allow for a more efficient use of the delivered drugs. This will result both in a reduction of the amount of required medication and a reduction of power required by the system 100.

### LIST OF REFERENCE SIGNS

100 block diagram of drug delivery system
112 motion sensing unit/3D acceleration sensor
113 data connection (wired or wireless)
120 control unit/processing and communication unit
122 control line (wired or wireless)
125 input device
126 output device
150 drug delivery device
152 feedback line
280 module comprising sensing unit, power supply, control unit and transmission unit
280a cross section of module 280
285a cross section of module comprising sensing unit, power supply, control unit and transmission unit
390 diagram depicting time dependency of tremor caused acceleration
391 acceleration amplitude in x-direction
392 absolute value of acceleration amplitude
395 diagram depicting Fourier Transform of acceleration in x-direction 691
450 drug delivery device/micro jet device
453 micro jet injector
454 skin contact sensors
455 USB port
456 temperature sensor
458 irritation sensor
459 strap
459a fixations means/Velcro fastener
512 motion sensing device/3D posture sensor/3D acceleration sensor
513 wireless data connection
550 drug delivery device/micro jet device
595 patient

1. A system for delivering at least one drug to the body of a patient (595), the system (100) comprising
a measurement device (112) for measuring at least one value of a parameter, which is indicative for the activity status of the patient (595),
a control unit (120), which is coupled to the measurement device (112) and which is adapted to determine a control signal based on the value of the parameter, and
a drug delivery device (150, 450, 550), which is coupled to the control unit (120) and which is adapted to deliver at least one drug to the patient's body (595) based on the control signal,
wherein the measurement device (112) comprises a plurality of acceleration sensors (512).

2. The system according to claim 1, wherein
the control unit is adapted to determine the control signal in real time.

3. The system according to claim 1, wherein
the control unit is adapted to determine the control signal based on the current time of day.

4. The system according to claim 1, further comprising
a memory (120) for storing a plurality of values of the parameter, which values are indicative for the activity status of the patient (595), wherein the values have been acquired at different times.

5. The system according to claim 1, further comprising
a unit (120) for evaluating the frequency distribution (395) of a signal provided by the acceleration sensors (112).

6. The system according to claim 1, wherein
the measurement device (112), the control unit (120) and/or the drug delivery device (150) are designed to be externally and mechanically coupled to a patient's body.

7. The system according to claim 1, wherein
the coupling between the measurement device (112), the control unit (120) and the drug delivery device (150) comprises a wireless connection.

8. The system according to claim 1, wherein
drug delivery device (150) is realized by a module, which can be spatially separated from the measurement device (112) and/or from the control unit (120).

9. The system according to claim 11, wherein
the drug delivery device (150) is a micro jet based device (450).

10. The system according to claim 11, wherein
the drug delivery device (150) is an iontophoretic device.

11. The system according to claim 1, wherein
the measurement device (112) comprises a magnetic field sensor.

12. The system according to claim 1, wherein
the measurement device (112) comprises a skin impedance sensor (754).

13. The system according to claim 1, wherein
the measurement device (112) comprises a heart rate sensor and/or a sweat sensor.

14. The system according to claim 1, further comprising
a communication unit (120), which is coupled to the control unit.

15. A method for delivering at least one drug to the body of a patient (595), the method comprising
measuring at least one value of a parameter by means of a plurality of acceleration sensors (512), which parameter is indicative for the activity status of the patient (595),
determining a control signal based on the value of the parameter, and
delivering drugs to the patient's body (595) based on the control signal.
